(19) United States
(12) Patent Application Publication
(10) Pub. No.: US 2011/0306888 A1
(43) Pub. Date: Dec. 15, 2011

(54) BLOOD PRESSURE MEASUREMENT DEVICE, BLOOD PRESSURE MEASUREMENT PROGRAM PRODUCT, AND BLOOD PRESSURE MEASUREMENT CONTROL METHOD

(75) Inventors: Tatsuya Kobayashi, Otsu-shi (JP); Tomonori Inoue, Kyoto-shi (JP)

(73) Assignee: OMRON HEALTHCARE CO., LTD., Kyoto (JP)

(21) Appl. No.: 13/213,300

(22) Filed: Aug. 19, 2011

Related U.S. Application Data
(63) Continuation of application No. PCT/JP2010/051697, filed on Feb. 5, 2010.

(30) Foreign Application Priority Data
Feb. 25, 2009 (JP) 2009-042606

(51) Int. Cl. A61B 5/0225

(52) U.S. Cl. 600/494

(57) ABSTRACT

When a start timing of an individual measurement control arrives, the pressurization control is started and a maximum blood pressure (blood pressure characteristic value) is estimated based on blood pressure characteristics information obtained during the pressurization control. The presence or absence of blood pressure fluctuation is determined based on the maximum blood pressure estimated this time and the (estimated) maximum blood pressure up to the previous times. The actual measurement process of the blood pressure is executed when determined that the blood pressure fluctuation is present. The actual measurement process is canceled when determined that the blood pressure fluctuation is absent.

### TECHNICAL FIELD

The present invention relates to blood pressure measurement devices, blood pressure measurement program products, and blood pressure measurement control methods, and in particular to a blood pressure measurement device, a blood pressure measurement program product, and a blood pressure measurement control method for executing measurement control of the blood pressure over a plurality of times and measuring the blood pressure in the depressurization process.

### BACKGROUND ART

A portable ABPM (Ambulatory Blood Pressure Monitoring) device (also known as “24 hour sphygmomanometer”) has been developed in recent years. In such a device, monitoring of blood pressure fluctuation, evaluation of cardiovascular disease development risk, or the like is carried out by executing the blood pressure measurement on a regular basis (carry out so-called interval measurement).

A device for automatically measuring the blood pressure when the oxygen saturation in the blood becomes abnormal is conventionally known (Japanese Unexamined Patent Publication No. 62-155829 (patent document 1)). A device for measuring the blood pressure from a change in physiological information is also known (Japanese Unexamined Patent Publication No. 2005-237472 (patent document 2)).

A technique of determining abnormal blood pressure lowering based on the area of the envelope curve within a pressure range lower than the average blood pressure is also known (Japanese Unexamined Patent Publication No. 7-303614 (patent document 3)). A technique of determining the presence or absence of a change in the blood pressure value by comparing the pulse wave amplitude in the range lower than the maximum blood pressure and the recorded pulse wave amplitude is also known (Japanese Unexamined Patent Publication No. 8-56911 (patent document 4)).
Patent Document 1: Japanese Unexamined Patent Publication No. 62-155829
Patent Document 2: Japanese Unexamined Patent Publication No. 2005-237472
Patent Document 3: Japanese Unexamined Patent Publication No. 7-303614
Patent Document 4: Japanese Unexamined Patent Publication No. 8-56911

### SUMMARY OF INVENTION

In techniques such as Japanese Unexamined Patent Publication No. 62-155829 and Japanese Unexamined Patent Publication No. 2005-237472 (patent documents 1 and 2), the change in blood pressure value (blood pressure fluctuation) may be captured, but the determination thereof is carried out independent from the usual measurement control and thus is inefficient.

The techniques of Japanese Unexamined Patent Publication No. 7-303614 and Japanese Unexamined Patent Publication No. 8-56911 (patent documents 3 and 4) relate to a device for measuring the pulse wave in the process of gradually compressing or a process of compressing and then gradually depressurizing the measurement site (e.g., upper arm) of the person to be measured, and hence, measurement error may occur if the person moves the body during the measurement. Thus, the person to be measured needs to maintain at rest as much as possible during the depressurization for every measurement control, which is a burden on the person to be measured.

Therefore, one or more embodiments of the present invention provides a blood pressure measurement device, a blood pressure measurement program product, and a blood pressure measurement control method capable of alleviating the burden of the person to be measured when automatically executing the measurement control of the blood pressure over a plurality of times.

According to one or more embodiments of the present invention, a blood pressure measurement device is a blood pressure measurement device for automatically executing a measurement control of a blood pressure over a plurality of times, the blood pressure measurement device including a cuff to be wrapped around a predetermined measurement site; and a control unit for carrying out the measurement control for every first period during a measurement mode. The control unit includes a timing determination unit for determining whether or not a first timing corresponding to the start of the first period has arrived, a pressurization control unit for carrying out a pressurization control of the cuff at a first speed when determined that the first timing has arrived, and a measurement processing unit for executing the actual measurement process of the blood pressure after the pressurization control. The measurement processing unit includes a depressurization control section for carrying out a depressurization control of the cuff at a second speed slower than the first speed, and a determination section for determining a blood pressure value of a person to be measured based on blood pressure characteristics information obtained during the depressurization control. The control unit also includes an estimation processing unit for estimating a blood pressure characteristic value representing a maximum blood pressure or a minimum blood pressure based on blood pressure characteristics information obtained during the pressurization control, and a fluctuation determination unit for determining presence or absence of blood pressure fluctuation based on a first blood pressure characteristic value estimated this time and a second blood pressure characteristic value up to previous time, and the measurement processing unit executes the actual measurement process when determined by the fluctuation determination unit that the blood pressure fluctuation is present.

According to one or more embodiments of the present invention, control unit cancels the actual measurement process when determined by the fluctuation determination unit that the blood pressure fluctuation is absent.

According to one or more embodiments of the present invention, storage unit for storing at least one blood pressure characteristic value estimated by the estimation processing unit is further arranged. The second blood pressure characteristic value represents a blood pressure characteristic value estimated the previous time, or an average value of blood pressure characteristic values for a predetermined number of times estimated in the past.

According to one or more embodiments of the present invention, the fluctuation determination unit determines that the blood pressure fluctuation is present when a difference of the first blood pressure characteristic value and the second blood pressure characteristic value is greater than or equal to a set value.

According to one or more embodiments of the present invention, the set value represents a value set by the user.

According to one or more embodiments of the present invention, the timing determination unit further determines whether or not a second timing corresponding to the start of a second period has arrived; the second period corresponds to plural multiples of the first period; and the measurement processing unit executes the actual measurement process regardless of the determination result by the fluctuation determination unit when determined that the second timing has arrived.

According to one or more embodiments of the present invention, a timing unit for counting a time corresponding to the first period and the second period is further arranged.

According to one or more embodiments of the present invention, a pressure detection unit for detecting a cuff pressure signal representing a pressure of the cuff is further arranged. The blood pressure characteristics information represent information of a pulse wave amplitude extracted by the cuff pressure signal.

According to one or more embodiments of the present invention, a blood pressure measurement program product is a blood pressure measurement program product for automatically executing a measurement control of a blood pressure over a plurality of times, the blood pressure measurement program product causing a computer to execute the steps of determining whether or not a first timing corresponding to the start of a first period has arrived; carrying out a pressurization control of a cuff at a first speed when determined that the first timing has arrived; estimating a blood pressure characteristic value representing a maximum blood pressure or a minimum blood pressure based on blood pressure characteristics information obtained during the pressurization control; determining presence or absence of blood pressure fluctuation based on a first blood pressure characteristic value estimated this time and a second blood pressure characteristic value up to previous time; carrying out a depressurization control of the cuff at a second speed slower than the first speed when determined that the blood pressure fluctuation is present; and determining a blood pressure value of a person to be measured based on blood pressure characteristics information obtained during the depressurization control.

According to one or more embodiments of the present invention, a blood pressure measurement control method relates to a blood pressure measurement control method for automatically executing a measurement control of a blood pressure over a plurality of times, the blood pressure measurement control method including the steps of determining whether or not a first timing corresponding to the start of a first period has arrived; carrying out a pressurization control of a cuff at a first speed when determined that the first timing has arrived; estimating a blood pressure characteristic value representing a maximum blood pressure or a minimum blood pressure based on blood pressure characteristics information obtained during the pressurization control; determining presence or absence of blood pressure fluctuation based on a first blood pressure characteristic value estimated this time and a second blood pressure characteristic value up to previous time; carrying out a depressurization control of the cuff at a second speed slower than the first speed when determined that the blood pressure fluctuation is present; and determining a blood pressure value of a person to be measured based on blood pressure characteristics information obtained during the depressurization control.

According to one or more embodiments of the present invention, the burden of the person to be measured in keeping rest for every measurement control can be reduced because the actual measurement process of the blood pressure is executed when determined that the blood pressure fluctuation is present.

Furthermore, in the blood pressure measurement device for measuring the blood pressure in the depressurization process, the blood pressure characteristic value (maximum blood pressure or average blood pressure) is estimated based on the blood pressure characteristic information obtained at the time of the pressurization control, and the presence or absence of the blood pressure fluctuation is determined based on the estimation result. Thus, the presence or absence of blood pressure fluctuation can be efficiently determined.

### BRIEF DESCRIPTION OF THE DRAWINGS

FIG. 1 is a perspective view of an outer appearance of a blood pressure measurement device according to one or more embodiments of the present invention.

FIG. 2 is a block diagram showing a hardware configuration of the blood pressure measurement device according to one or more embodiments of the present invention.

FIG. 3 is a block diagram showing a function configuration of the blood pressure measurement device according to one or more embodiments of the present invention.

FIG. 4 is a flowchart showing a process (continuous measurement process) executed during the measurement mode by the blood pressure measurement device according to one or more embodiments of the present invention.

FIGS. 5A and 5B are views describing one example of a blood pressure estimating process (step S9 of FIG. 4) according to one or more embodiments of the present invention.

FIG. 6 is a flowchart showing the actual measurement process (step S18 of FIG. 4) of the blood pressure according to one or more embodiments of the present invention.

FIG. 7 is a view showing a result display example when the actual measurement process is executed according to one or more embodiments of the present invention.

FIG. 8 is a view showing a result display example when the actual measurement process is not executed according to one or more embodiments of the present invention.

FIG. 9 is a view showing a data structure example of the blood pressure information temporarily recorded in the memory while the process (measurement mode) shown in FIG. 4 is being executed.

FIGS. 10A and 10B are views showing a data structure example of measurement result data according to one or more embodiments of the present invention.

FIG. 11 is a view showing a concept of the interval measurement in a typical blood pressure measurement device.

FIGS. 12A and 12B are views showing the concept of the interval measurement in the blood pressure measurement device according to one or more embodiments of the present invention.

### DETAILED DESCRIPTION OF THE INVENTION

Embodiments of the present invention will be described in detail with reference to the drawings. The same reference numerals are denoted for the same or corresponding portions in the figures, and the description thereof will not be repeated.

A blood pressure measurement device (hereinafter referred to as “sphygmomanometer”) according to one or more embodiments of the present invention automatically executes the measurement control of the blood pressure over a plurality of times. The sphygmomanometer according to one or more embodiments of the present invention may be an ABPM device or a blood pressure monitor.

<Outer Appearance and Configuration>

First, the outer appearance and the configuration of the sphygmomanometer according to one or more embodiments of the present invention will be described.

(Regarding Outer Appearance)

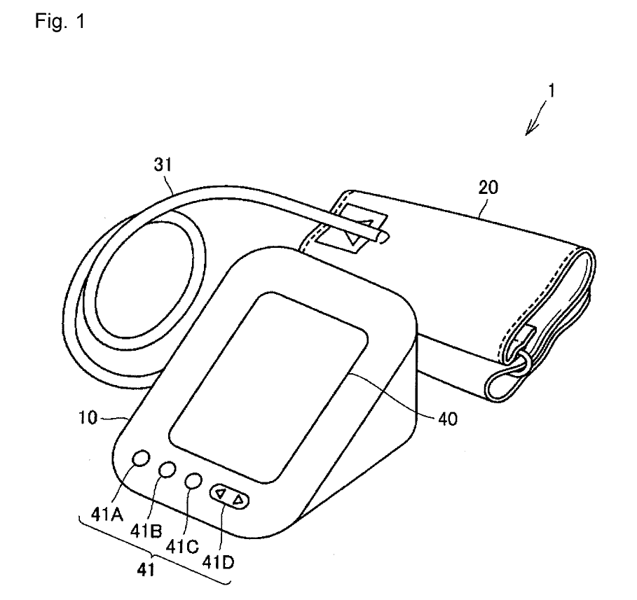

With reference to FIG. 1, a sphygmomanometer 1 includes a main body 10, a cuff 20 that can be wrapped around a predetermined measurement site (e.g., upper arm) of a person to be measured, and an air tube 31 for connecting the main body 10 and the cuff 20.

A display unit 40 configured by liquid crystal, or the like, and an operation unit 41 for accepting instructions from the user (medical staff such as doctor, or person to be measured) are arranged on the surface of the main body 10.

The operation unit 41 includes a power supply switch 41A for accepting input of the instruction to turn ON or OFF the power supply, a start switch 41B for accepting the instruction to start the measurement, a stop switch 41C for accepting the instruction to stop the measurement, and a set switch 41D for accepting the instruction for various types of setting processes and reading of storage values.

(Regarding Hardware Configuration)

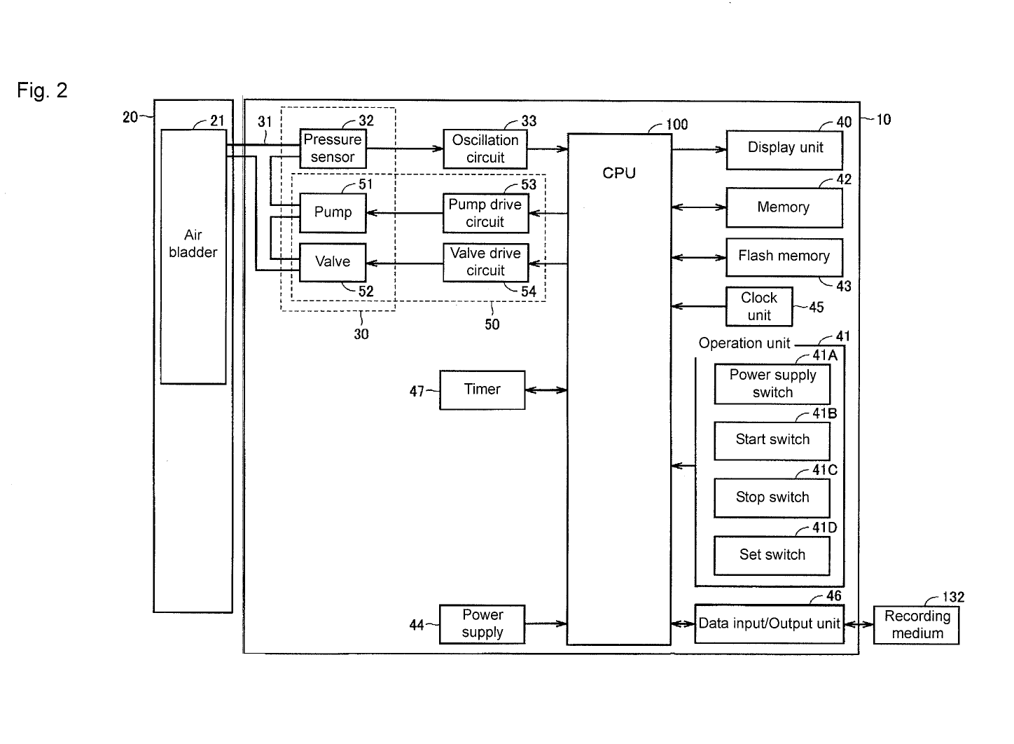

With reference to FIG. 2, the cuff 20 of the sphygmomanometer 1 includes an air bladder 21. The air bladder 21 is connected to an air system 30 through an air tube 31.

In addition to the display unit 40 and the operation unit 41, the main body 10 includes the air system 30, a CPU (Central Processing Unit) 100 for controlling each unit in a concentrated manner and performing various types of calculation processes, a memory 42 for storing various types of programs and data, a nonvolatile memory (e.g., flash memory) 43 for storing the measured blood pressure, a power supply 44 for supplying power to the CPU 100 or the like, a clock unit 45 for timing the current date and time, or the like, a data input/output unit 46 for accepting input of data from the outside, and a timer 47 for performing the timing operation.

The air system 30 includes a pressure sensor 32 for detecting the pressure (cuff pressure) of the air bladder 21, a pump 51 for supplying air to the air bladder 21 to pressurize the cuff pressure, and a valve 52 to be opened and closed to exhaust or enclose the air of the air bladder 21.

The main body 10 further includes an oscillation circuit 33, a pump drive circuit 53, and a valve drive circuit 54 in relation to the air system 30.

The pressure sensor 32 is a capacitance type pressure sensor in which the capacitance value changes by the cuff pressure. The oscillation circuit 33 outputs a signal having an oscillating frequency corresponding to the capacitance value of the pressure sensor 32 to the CPU 100. The CPU 100 converts the signal obtained from the oscillation circuit 33 to pressure and detects the pressure. The pump drive circuit 53 controls the drive of the pump 51 based on a control signal provided from the CPU 100. The valve drive circuit 54 performs the open/close control of the valve 52 based on a control signal provided from the CPU 100.

The pump 51, the valve 52, the pump drive circuit 53, and the valve drive circuit 54 configure an adjustment unit 50 for adjusting the cuff pressure. The configuration of the adjustment unit 50 is not limited thereto as long as the cuff pressure can be adjusted.

The data input/output unit 46 reads and writes programs and data with respect to a removable recording medium 132. Instead, the data input/output unit 46 may transmit and receive programs and data with an external computer (not shown) through a communication line.

The air bladder 21 is arranged in the cuff 20, but the fluid supplied to the cuff 20 is not limited to air, and may be liquid or gel. Alternatively, it is not limited to fluid, and may be uniform fine particles such as micro-beads.

(Regarding Function Configuration)

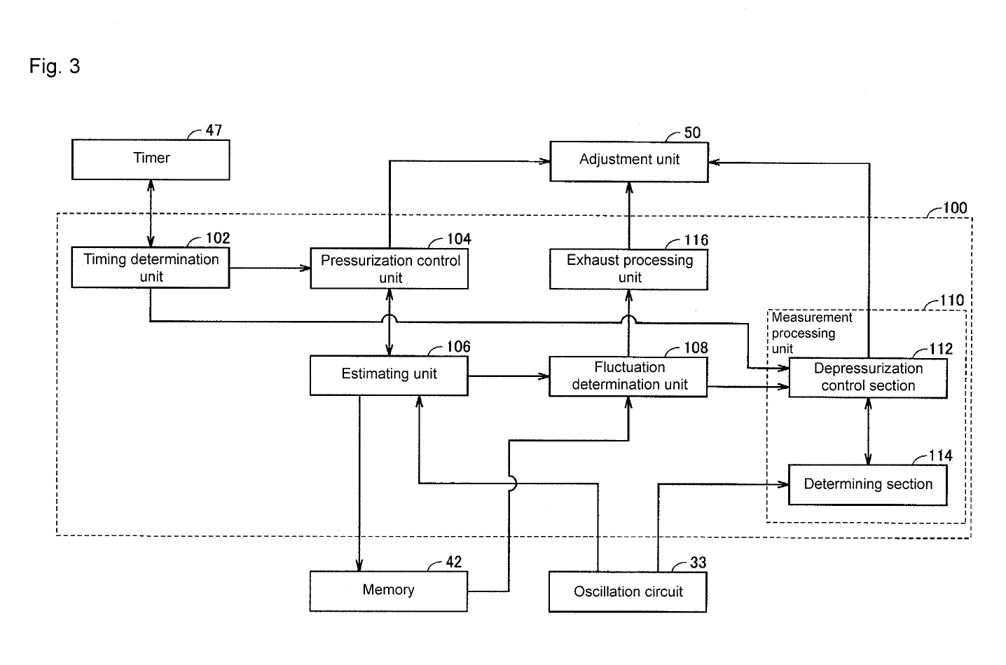

With reference to FIG. 3, the CPU 100 performs the measurement control (hereinafter referred to as “individual measurement control”) of the blood pressure for every first period (hereinafter referred to as “interval period 1”) during the measurement mode. The CPU 100 includes a timing determination unit 102, a pressurization control unit 104, an estimating unit 106, a fluctuation determination unit 108, a measurement processing unit 110, and an exhaust processing unit 116 for such function.

Only the peripheral hardware that directly exchanges signals with each unit of the CPU 100 is shown in FIG. 3 to simplify the description.

The timing determination unit 102 determines whether or not a first timing corresponding to the start of the interval period 1 has arrived using the timer 47. Whether or not a second timing corresponding to the start of a second period (hereinafter referred to as “interval period 2”) has arrived is also determined.

According to one or more embodiments of the present invention, the “interval period 1” represents a measurement interval in a device that performs a so-called interval measurement existing from the prior art. That is, the interval period 1 represents the interval of the start timings of two successive individual measurement controls. The “interval period 2” corresponds to a plural multiple (e.g., 5 times) of the interval period 1. That is, the interval period 2 represents the interval from the start timing of the first individual measurement control to the start timing of the fifth individual measurement control.

The individual measurement control executed for every interval period 1 always includes the pressurization control, but may not include the actual measurement process (actual measurement process of blood pressure value), to be described later.

The timing determination unit 102 outputs a pressurization command to the pressurization control unit 104 when determined that the current is the first timing. The timing determination unit 102 outputs a measurement command to the measurement processing unit 110 when determined that the current is the second timing.

The pressurization control unit 104 is connected to the adjustment unit 50, and performs the pressurization control of the cuff. The pressurization control is executed when determined that the first timing (similarly second timing) has arrived. As hereinafter described in detail, the sphygmomanometer 1 according to one or more embodiments of the present invention measures the blood pressure in the depressurization process, and hence, the speed of pressurization is a speed (sufficiently) faster than the speed of depressurization.

The estimating unit 106 estimates the maximum blood pressure based on the information of the pulse wave amplitude obtained by the oscillation circuit 33 during the pressurization control. The data of the estimated maximum blood pressure (estimated maximum blood pressure) is output to the fluctuation determination unit 108. The estimated blood pressure characteristic value is not limited to the maximum blood pressure, and may be an average blood pressure. The data of the estimated maximum blood pressure is stored in a predetermined region of the memory 42.

The fluctuation determination unit 108 determines the presence or absence of the blood pressure fluctuation based on the estimated maximum blood pressure of this time and the estimated maximum blood pressure of the previous time stored in the memory 42. More specifically, determination is made that there is blood pressure fluctuation when the difference between the estimated maximum blood pressure of this time and the estimated maximum blood pressure of the previous time is greater than or equal to a set value. If determined that there is blood pressure fluctuation, this is notified to the measurement processing unit 110. If determined that there is no blood pressure fluctuation, this is notified to the exhaust processing unit 116.

The set value may be a value set by the user. Thus, a criterion value of the blood pressure fluctuation may be a value corresponding to the characteristics of the blood pressure change of the person to be measured.

According to one or more embodiments of the present invention, comparison is made with the estimated maximum blood pressure of the previous time to determine the presence or absence of the blood pressure fluctuation, but it is not restrictive as long as it is a value estimated during the measurement mode of this time. For example, the average value for a predetermined immediate number of times (e.g. three times) may be used for the comparison with the estimated maximum blood pressure of this time.

Alternatively, in the individual measurement control of the previous time, the maximum blood pressure of the actual measurement in the individual measurement control of the previous time may be the target of comparison if the blood pressure is actually measured by the measurement processing unit 110, to be described later.

The measurement processing unit 110 executes the actual measurement process of the blood pressure. The actual measurement process is executed when determined that there is blood pressure fluctuation by the fluctuation determination unit 108, or when determined that the start timing of the individual measurement control of this time is the second timing (start timing of the individual measurement control corresponding to the interval period 2) by the timing determination unit 102.

The measurement processing unit 110 includes a depressurization control section 112 and a determination section 114 as functions for the actual measurement process. The depressurization control section 112 is connected to the adjustment unit 50, and performs the depressurization control of the cuff 20. The determination section 114 has a function for determining the blood pressure value of the person to be measured based on the information of the pulse wave amplitude obtained by the oscillation circuit 33 during the depressurization control.

The exhaust processing unit 116 rapidly exhausts the air in the air bladder 21. If determined that there is no blood pressure fluctuation by the fluctuation determination unit 108, the actual measurement process by the measurement processing unit 110 is canceled and the exhausting process is carried out.

The operation of each function block may be realized by executing the software stored in the memory 42 or at least one may be realized with hardware.

<Regarding Operation>

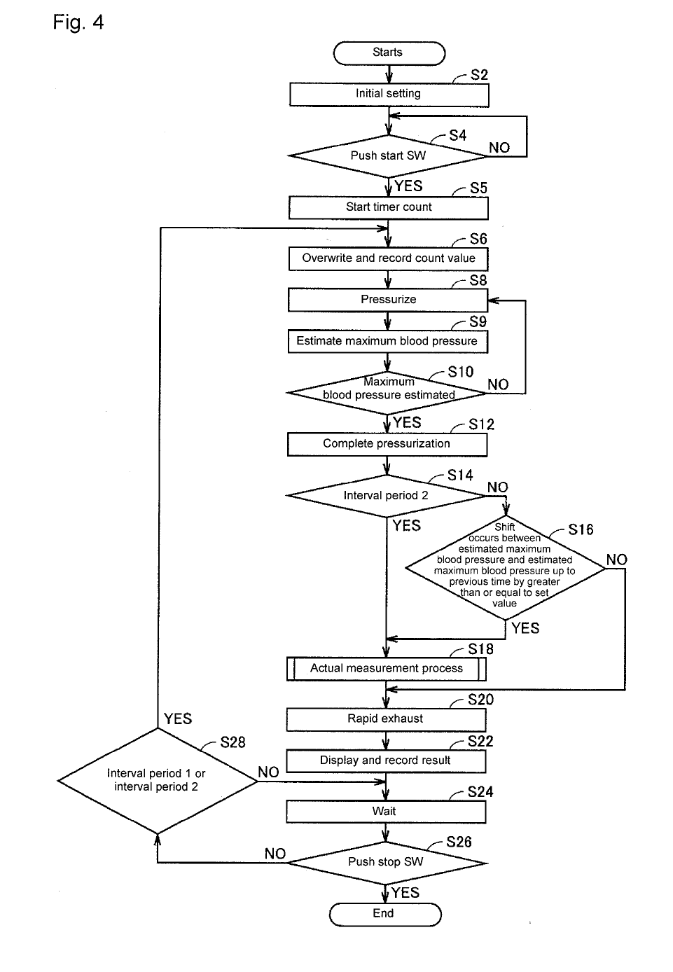

With reference to the flowchart of FIG. 4, the process (hereinafter referred to as “continuous measurement process”) executed during the measurement mode by the sphygmomanometer 1 according to one or more embodiments of the present invention will now be described. The processes shown in the flowchart of FIG. 4 are stored in the memory 42 as a program in advance, where the function of the continuous measurement process is realized when the CPU 100 reads out and executes this program.

With reference to FIG. 4, when the power supply switch 41A is pushed, the CPU 100 performs the initialization process (step S2). Specifically, a predetermined region of the memory 42 is initialized, the air of the air bladder 21 is exhausted, and the 0 mmHg correction of the pressure sensor 32 is carried out. The timer 47 is then reset.

When the start switch 41B is pushed (YES in step S4), the sphygmomanometer 1 is transitioned to the measurement mode. According to one or more embodiments of the present invention, the actual measurement process is always executed immediately after the transition to the measurement mode, that is, in the first individual measurement control.

After transitioning to the measurement mode, the timing determination unit 102 first starts the count of the timer 47 (step S5). At the same time, the current count value of the timer 47 is temporarily stored (step S6). In the first individual measurement control, “0” is temporarily stored as the count value. The count value stored in this process represents the elapsed time to the start timing of the individual measurement control of this time after transitioning to the measurement mode. The count value may be overwritten and stored in the predetermined region of the memory 42.

Thereafter, the pressurization control unit 104 pressurizes the cuff 20 (step S8). Specifically, the valve 52 is closed, and the control is carried out to pressurize the cuff 20 at high speed (e.g., 30 mmHg/s) by means of the pump 51.

During the pressurization, the estimating unit 106 estimates the maximum pressure (step S9). The pressurization is continued until the estimation of the maximum blood pressure is completed (NO in step S10). The estimation of the maximum blood pressure can be realized through a known method.

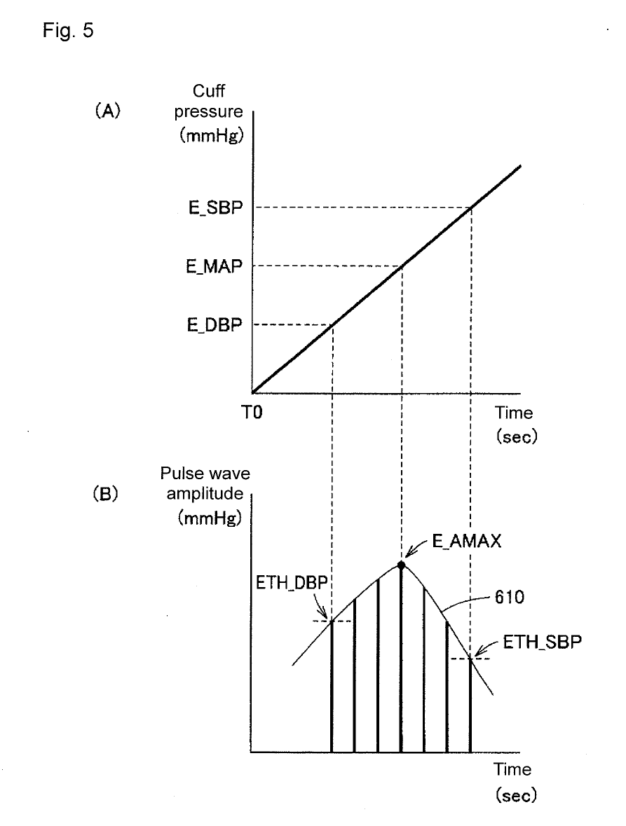

One example of a method for estimating the maximum blood pressure will be shown with reference to FIG. 5.

FIG. 5A shows the cuff pressure (unit: mmHg) to be gradually pressurized along the time axis, and FIG. 5B partially shows the pulse wave amplitude (unit: mmHg) superimposed on the cuff pressure along the same time axis.

The estimating unit 106 extracts the pulse wave amplitude superimposed on the cuff pressure based on the output from the oscillation circuit 33, and detects a maximum value E_AMAX of the pulse wave amplitude. The cuff pressure corresponding to the maximum value E_AMAX of the pulse wave amplitude is specified as an estimated average blood pressure E_MAP.

The estimating unit 106 calculates a threshold value ETH_SBP that is a value obtained by multiplying a predetermined constant (e.g., 0.5) on the maximum value E_AMAX. A point where an envelope curve 610 of the pulse wave amplitude and the threshold value ETH_SBP intersect is extracted in the process of increasing the cuff pressure to higher than the estimated average blood pressure (E_MAP), so that the cuff pressure corresponding to such point is determined as the estimated maximum blood pressure E_SBP.

A point where a value ETH_DBP obtained by multiplying a predetermined constant (e.g., 0.7) to the maximum point E_AMAX and the envelope curve 610 of the pulse wave amplitude intersect may be extracted, and the cuff pressure (lower than estimated average blood pressure E_MAP) corresponding to such point may be determined as the estimated minimum blood pressure E_DBP.

With reference again to FIG. 4, when the maximum blood pressure is estimated by the estimating unit 106 (YES in step S10), the drive of the pump 51 is stopped and the pressurization is completed (step S12). The estimating unit 106 stores the estimated maximum blood pressure in a predetermined region of the memory 42. A data structure example of the blood pressure information stored in the memory 42 during the continuous measurement process will be described later.

According to one or more embodiments of the present invention, the pressurization is terminated when the maximum blood pressure is estimated, but the maximum blood pressure may be estimated (based on the pulse wave amplitude information obtained during pressurization) after the pressurization is terminated.

The timing determination unit 102 determines whether or not this time is the interval period 2 (step S14). Determination may be made as always corresponding to the interval period 2 immediately after the start (count value=0 of timer 47).

If determined as corresponding to the interval period 2 (YES in step S14), the process proceeds to step S18. If determined as not corresponding to the interval period 2 (NO in step S14), the process proceeds to step S16.

In step S16, the fluctuation determination unit 108 determines whether or not a shift occurred between the maximum blood pressure of this time estimated in step S9 and the estimated maximum blood pressure of the previous time by a set value or greater, that is, whether or not the blood pressure fluctuation occurred.

If determined that the blood pressure fluctuation occurred (YES in step S16), the process proceeds to step S18. If not (NO in step S16), step S18 is skipped and the process proceeds to step S20.

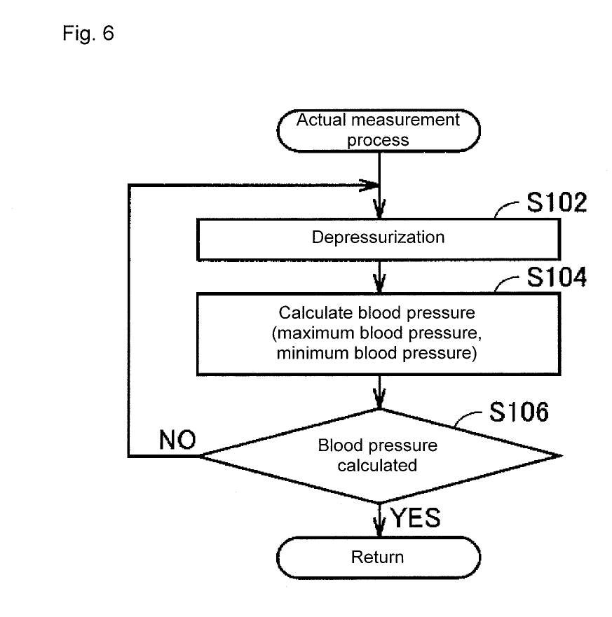

In step S18, the measurement processing unit 110 executes the actual measurement process of the blood pressure. The details of the actual measurement process are shown in the flowchart of FIG. 6.

With reference to FIG. 6, the depressurization control section 112 of the measurement processing unit 110 performs a control of gradually depressurizing the cuff 20 at a predetermined speed (e.g., 4 mmHg/s) by controlling the open amount of the valve 52 (step S102). The depressurization speed is a speed sufficiently slower than the pressurization speed to obtain an accurate blood pressure value.

During depressurization, the determination section 114 of the measurement processing unit 110 executes the calculation process of the blood pressure (step S104). According to one or more embodiments of the present invention, the maximum blood pressure and the minimum blood pressure are calculated based on the oscillometric method (according to the theory similar to the blood pressure estimating process described above).

The depressurization is continued until the calculation of the blood pressure is completed (NO in step S106).

After the maximum blood pressure and the minimum blood pressure are calculated (determined (YES in step S106)), they are stored in time series in a predetermined region of the memory 42. The process then proceeds to step S20 of the main routine.

According to one or more embodiments of the present invention, the depressurization is terminated when the maximum blood pressure and the minimum blood pressure are determined, but the maximum blood pressure and the minimum blood pressure may be calculated (based on pulse wave amplitude information obtained during depressurization) after the depressurization is terminated.

In step S20, the exhaust processing unit 116 controls the valve drive circuit 54 to completely open the valve 52 to rapidly exhaust the air (step S20).

The result of the individual measurement control of this time is then displayed and recorded (step S22), and the individual measurement control of this time is terminated.

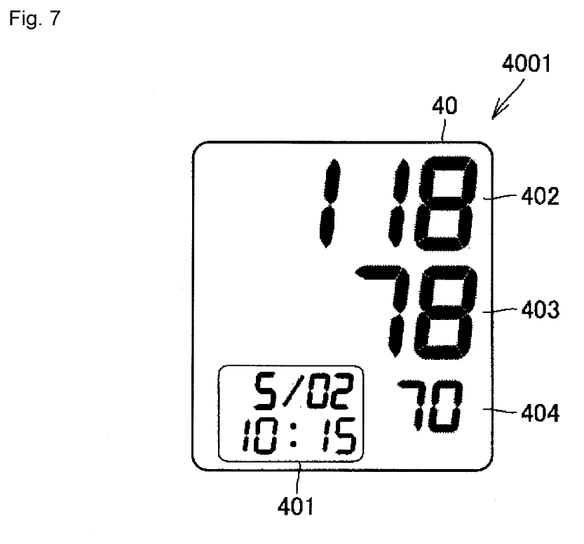

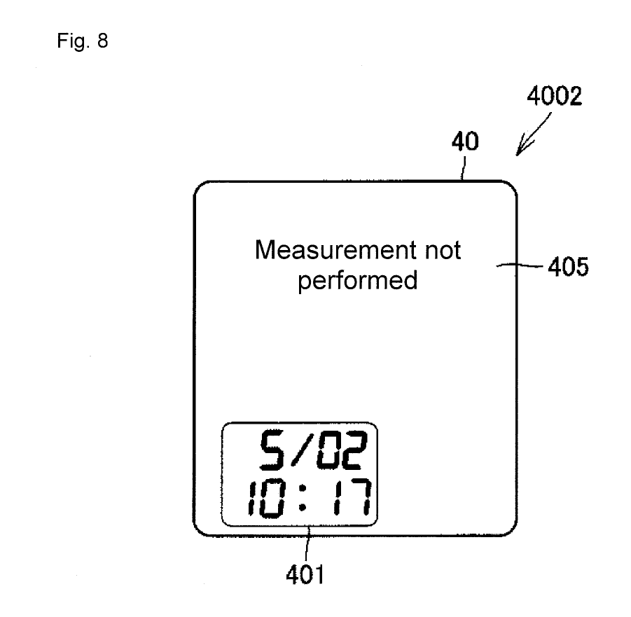

If the actual measurement process is executed, a screen 4001 as shown in FIG. 7 or the like is displayed. If the actual measurement process is not executed, a screen 4002 as shown in FIG. 8 or the like is displayed.

With reference to FIG. 7, the measurement date and time is displayed in a region 401 of the screen 4001, and the maximum blood pressure value and the minimum blood pressure value calculated in step S104 of FIG. 6 are displayed in the regions 402 and 403, respectively. The number of pulses that can be calculated through a known method may be displayed in the region 404.

With reference to FIG. 8, a message 405 “measurement not performed” may be displayed in the screen 4002. The user is thus notified that the actual measurement process is not executed in the individual measurement control of this time.

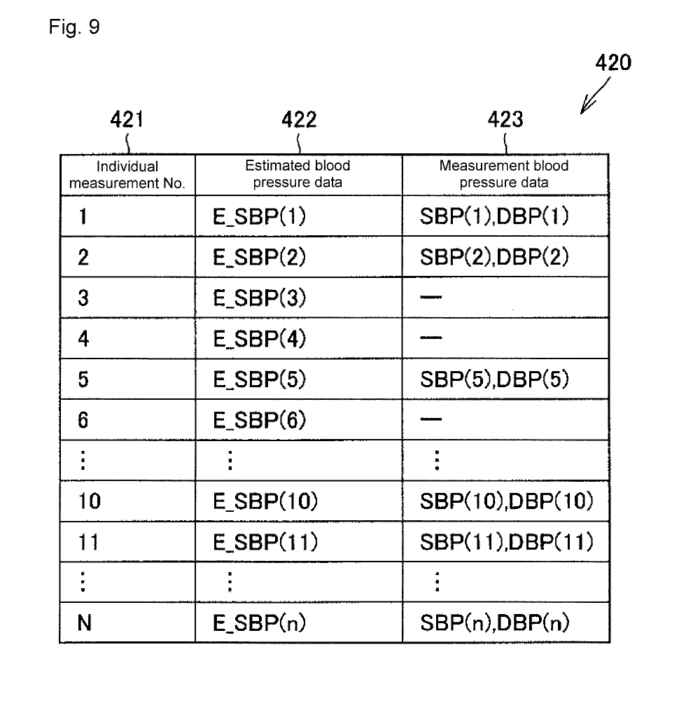

FIG. 9 is a view showing a data structure example of the blood pressure information 420 recorded in a predetermined region of the memory 42 during the continuous measurement process (measurement mode).

With reference to FIG. 9, the blood pressure information 420 includes a region 421 for storing “individual measurement number”, a region 422 for storing “estimated blood pressure data”, and a region 423 for storing “measurement blood pressure data”.

The individual measurement number is an identification number for identifying each individual measurement, and is given by being incremented one at a time at the start of pressurization (between steps S6 and S8).

The estimated blood pressure (estimated maximum blood pressure) and measurement blood pressure (estimated maximum blood pressure and minimum blood pressure) are recorded in correspondence with each individual measurement number. The estimated blood pressure is always recorded for every individual measurement number, but the measurement blood pressure is recorded only when the actual measurement process is executed.

FIG. 9 shows an example in which the interval period 2 is five times the interval period 1, and the actual measurement process is always executed once every five times.

The structure of the blood pressure information stored during the measurement mode is not limited to that shown in FIG. 9. For instance, according to one or more embodiments of the present invention, only the estimated blood pressure data for the previous time may be stored because the estimated blood pressure for the previous time is used to determine the presence or absence of the blood pressure fluctuation.

With reference again to FIG. 4, the CPU 100 enters the waiting state after the individual measurement control for one time is finished (step S24). While waiting, the CPU 100 determines whether or not the stop switch 41C is pushed (step S26). If determined that the stop switch 41C is not pushed (NO in step S26), the process proceeds to step S28.

In step S28, the timing determination unit 102 i) determines whether or not the elapsed time from the start timing of the individual measurement control of immediately before is equal to the time of the interval period 1, and ii) determines whether or not the elapsed time from when determined as the start timing (second timing) of the interval period 2 the previous time is equal to the period of the interval period 2.

The determination of i) can be made by determining whether or not the time obtained from the difference between the current count value of the timer 47 and the count value stored in step S6 matches the time of the interval period 1.

The determination of ii) can be made by determining whether or not the current count value of the timer 47 matches the multiples of the interval period 2. For instance, determination is made on whether or not the count value of the timer 47 is 5 minutes, 10 minutes, and 15 minutes, assuming the interval period 2 is 5 minutes.

The method of determining the interval periods 1 and 2 is not limited to the above. For instance, another timer (not shown) may be arranged so that the timer 47 is dedicated for the determination of the interval period 1 and another timer is dedicated for the determination of the interval period 2. In this case, the timer 47 is reset if determined as the interval period 1 and another timer is reset if determined as the interval period 2 in step S28.

Alternatively, the timer 47 or the like may not be arranged, the start time of the continuous measurement process and the start time of the individual measurement process may be stored in the internal memory by the time information (date, hour, minutes, seconds) output by the clock unit 45, and each interval period may be determined based on the time of the specific timing and the current time.

The individual measurement number given for every individual measurement control may be used, so that determination may be made as the interval period 2 when the individual measurement number is 1, 6, 11 (every “5”).

According to one or more embodiments of the present invention, the start timing (second timing) of the interval period has been described as overlapping the first timing (start timing of interval period 1), but this is not the sole case.

If determined that the current timing does not correspond to the interval period 1 or 2 (NO in step S28), the process returns to step S24 and waits.

If determined that the current timing corresponds to the interval period 1 or 2 (YES in step S28), the process returns to step S6, and the individual measurement control described above is repeated.

If determined that the stop switch 41C is pushed in step S26 (YES in step S26), the CPU 100 stores the result of the series of continuous measurement processes in the flash memory 43 as measurement data, and terminates the present process.

<Regarding Data Structure Example of Measurement Result Data>

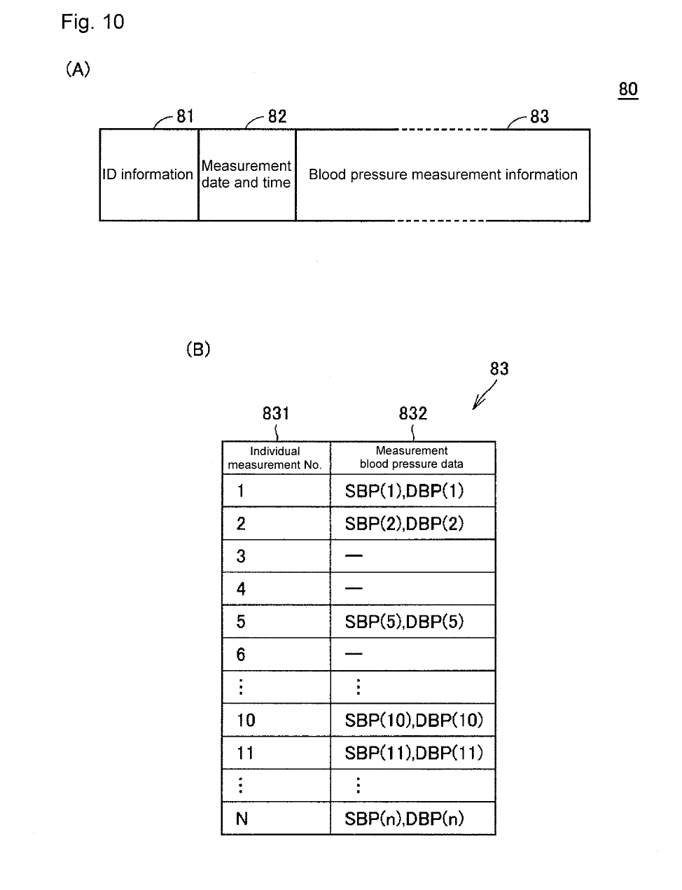

The structure example of the measurement result data stored in the flash memory 43 is shown in FIGS. 10A and 10B for every series of continuous measurement processes.

With reference to FIG. 10A, each measurement result data 80 stored in the flash memory 43 includes three fields 81 to 83 “ID information”, “measurement date and time”, and “blood pressure information” by way of example. Outlining the content of each field, the “ID information” field 81 stores the identification number and the like for specifying each measurement result data, and the “recording date and time” field 82 stores information such as measurement start date and time, and measurement period of each measurement result data timed by the clock unit 45. The “blood pressure information” field 83 stores the blood pressure data for every individual measurement process.

FIG. 10B is a view showing a data structure example of the blood pressure information field 83 included in the measurement result data.

With reference to FIG. 10B, the blood pressure information field 83 includes a region 831 for storing “individual measurement number” and a region 832 for storing “blood pressure data”. The region 831 and the region 832 correspond to the region 421 and the region 423 of the blood pressure information 420 shown in FIG. 9, respectively. That is, the estimated blood pressure data of the three items of the blood pressure information 420 shown in FIG. 9 is used only in the continuous measurement process, and thus may not be included in the information stored in the flash memory 43.

According to one or more embodiments of the present invention, the blood pressure information as shown in FIG. 10B is stored, but the information same as the blood pressure information 420 shown in FIG. 9 may be included in the blood pressure information field 83. In this case, the estimated maximum blood pressure is always recorded even if the actual measurement process is not executed, and hence, the approximate blood pressure value can be grasped.

The data of the measurement date and time (e.g., date, hour, minute of start of measurement control) may be further recorded for every individual measurement number.

According to one or more embodiments of the present invention, when the blood pressure is measured in each individual measurement control, it is once stored in the memory 42, and the necessary data are copied to the flash memory 43 when the series of continuous measurement processes are finished. However, the results may be directly stored in the flash memory 43 every time the blood pressure is measured in each individual measurement control.

The effects obtained by carrying out the operations described above will be described below.

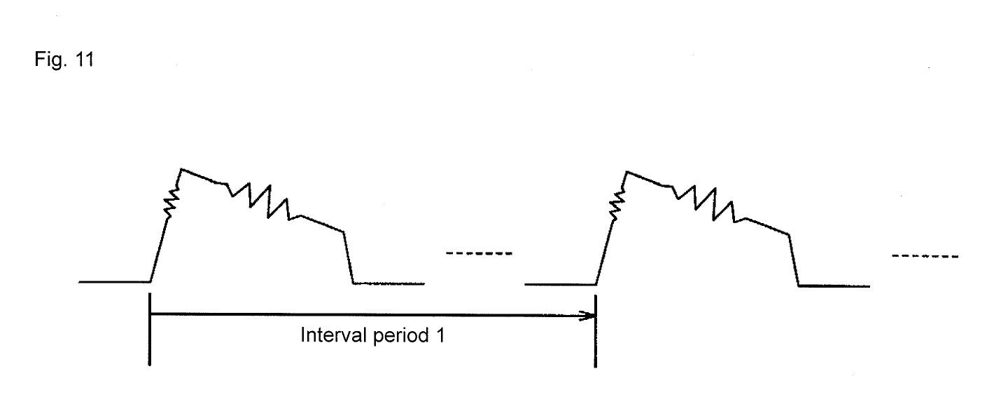

FIG. 11 shows the concept of interval measurement in a typical sphygmomanometer. With reference to FIG. 11, the blood pressure measurement (actual measurement process according to one or more embodiments of the present invention) is executed for every predetermined interval period (correspond to interval period 1 according to one or more embodiments of the present invention) in the typical sphygmomanometer.

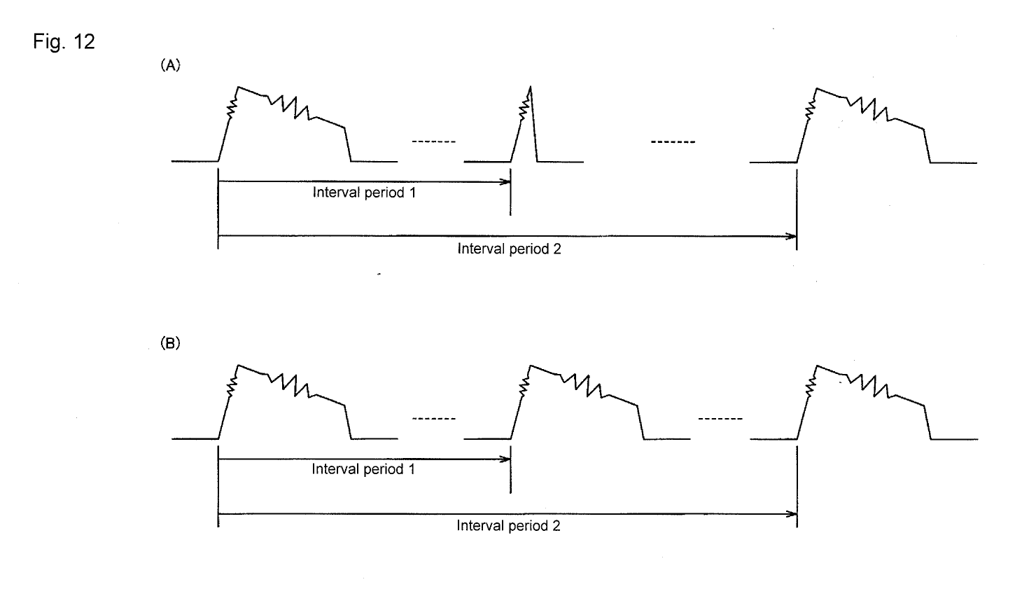

FIGS. 12A and 12B show the concept of the interval measurement in the sphygmomanometer 1 according to one or more embodiments of the present invention.

According to one or more embodiments of the present invention, the pressurization control is executed every interval period 1. However, if determined that there is no fluctuation (smaller than set value) in the estimated maximum blood pressure during the pressurization, the air is rapidly exhausted without performing the actual measurement process, as shown in FIG. 12A. If determined that there is fluctuation in the estimated maximum blood pressure during the pressurization, the actual measurement process is executed, as shown in FIG. 12B.

Therefore, according to one or more embodiments of the present invention, the physical or psychological burden of the person to be measured to be resting can be reduced for every interval period 1.

Furthermore, the fluctuation of the blood pressure is determined using the pulse wave amplitude information in the pressurization control, which is essential for the actual measurement process. Thus, the blood pressure fluctuation can be efficiently determined compared to when separately executing the process of determining the blood pressure fluctuation before the individual measurement control.

In the sphygmomanometer for performing interval measurement such as a blood pressure monitor, it is important to see how the blood pressure value is changing rather than what the blood pressure value is each time. In the sphygmomanometer 1 according to one or more embodiments of the present invention, the actual measurement (actual measurement process) of the blood pressure value is always carried out if there is blood pressure fluctuation, and hence, the original function in such a device will not be affected.

Furthermore, the change in blood pressure can be accurately recognized because the maximum blood pressure (estimated value) assumed to be important in blood pressure management is used for the determination of the blood pressure fluctuation.

Moreover, according to one or more embodiments of the present invention, the actual measurement process is always executed for every interval period 2 regardless of the presence or absence of the blood pressure fluctuation. As a result, according to one or more embodiments of the present invention, the burden of the person to be measured can be reduced without affecting the functions of the ABPM device or the blood pressure monitor.

According to one or more embodiments of the present invention, the estimation and measurement of the blood pressure are carried out according to the oscillometric method, but it is not limited to the oscillometric method as long as the blood pressure can be estimated based on the blood pressure characteristic information obtained at the time of pressurization, and the blood pressure can be measured based on the blood pressure characteristic information obtained at the time of depressurization. For instance, the estimation and measurement of the blood pressure may be carried out according to the Korotkov's sound method.

The function of the blood pressure measurement method (continuous blood pressure measurement process shown in FIG. 4) carried out by the CPU of the sphygmomanometer according to one or more embodiments of the present invention may be executed by an information processing device such as a personal computer. Such function may be provided as a program. Such program may be provided as a program product by being recorded on an optical medium such as a CD-ROM (Compact Disc-ROM), or a computer readable non-transitory recording medium such as a memory card. The program may be provided by being downloaded through a network.

The program according to one or more embodiments of the present invention may be for calling out the necessary module at a predetermined timing in a predetermined array and executing the process of the program modules provided as one part of the operating system (OS) of the computer. In this case, the relevant module is not included in the program itself and is operated cooperatively with the OS to execute the process. The program according to one or more embodiments of the present invention can also include the program that does not include such a module.

The program according to one or more embodiments of the present invention may be provided by being incorporated in one part of another program. In this case as well, the module included in another program is not included in the program itself and is operated cooperatively with another program to execute the process. The program according to one or more embodiments of the present invention can also include the program incorporated in another program.

The program product to be provided is installed in a program storage unit such as a hard disc, and executed. The program product includes the program itself and the storage medium in which the program is recorded.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 blood pressure measurement system (sphygmomanometer)
10 main body
20 cuff
21 air bladder
30 air system
31 air tube
32 pressure sensor
33 oscillation circuit
40 display unit
41 operation unit
41A power supply switch
41B start switch
41C stop switch
41D set switch
42 memory
43 flash memory
44 power supply
45 clock unit
46 data input/output unit
47 timer
50 adjustment unit
51 pump
52 valve
53 pump drive circuit
54 valve drive circuit
80 measurement result data
100 CPU
102 timing determination unit
104 pressurization control unit
106 estimating unit
108 fluctuation determination unit
110 measurement processing unit
112 depressurization control section
114 determination section
116 exhaust processing unit
132 recording medium

1. A blood pressure measurement device for automatically executing a measurement control of a blood pressure over a plurality of times, the blood pressure measurement device comprising:
a cuff to be wrapped around a predetermined measurement site; and
a control unit that carries out the measurement control for every first period during a measurement mode,
wherein the control unit comprises:
a timing determination unit that determines whether or not a first timing corresponding to a start of the first period has arrived;
a pressurization control unit that carries out a pressurization control of the cuff at a first speed when determined that the first timing has arrived;
an estimation processing unit that estimates a blood pressure characteristic value representing a maximum blood pressure or a minimum blood pressure based on blood pressure characteristics information obtained during the pressurization control;
a fluctuation determination unit that determines presence or absence of a blood pressure fluctuation based on a first blood pressure characteristic value estimated this time and a second blood pressure characteristic value up to a previous time; and
a measurement processing unit that executes an actual measurement process of the blood pressure after the pressurization control when determined by the fluctuation determination unit that the blood pressure fluctuation is present, and
wherein the measurement processing unit comprises:
a depressurization control section that carries out a depressurization control of the cuff at a second speed slower than the first speed; and
a determination section that determines a blood pressure value of a person to be measured based on blood pressure characteristics information obtained during the depressurization control.

2. The blood pressure measurement device according to claim 1, wherein the control unit cancels the actual measurement process when determined by the fluctuation determination unit that the blood pressure fluctuation is absent.

3. The blood pressure measurement device according to claim 1, further comprising:
a storage unit that stores at least one blood pressure characteristic value estimated by the estimation processing unit,
wherein the second blood pressure characteristic value represents a blood pressure characteristic value estimated the previous time, or an average value of blood pressure characteristic values for a predetermined number of times estimated in the past.

4. The blood pressure measurement device according to claim 1, wherein the fluctuation determination unit determines that the blood pressure fluctuation is present when a difference of the first blood pressure characteristic value and the second blood pressure characteristic value is greater than or equal to a set value.

5. The blood pressure measurement device according to claim 4, wherein the set value represents a value set by a user.

6. The blood pressure measurement device according to claim 1,
wherein the timing determination unit further determines whether or not a second timing corresponding to a start of a second period has arrived,
wherein the second period corresponds to plural multiples of the first period, and
wherein the measurement processing unit executes the actual measurement process regardless of a determination result by the fluctuation determination unit when determined that the second timing has arrived.

7. The blood pressure measurement device according to claim 6, further comprising a timing unit that counts a time corresponding to the first period and the second period.

8. The blood pressure measurement device according to claim 1, further comprising:
a pressure detection unit that detects a cuff pressure signal representing a pressure of the cuff,
wherein the blood pressure characteristics information represent information of a pulse wave amplitude extracted by the cuff pressure signal.

9. A blood pressure measurement program product for automatically executing a measurement control of a blood pressure over a plurality of times, the blood pressure measurement program product causing a computer to execute the steps of:
determining whether or not a first timing corresponding to a start of a first period has arrived;
carrying out a pressurization control of a cuff at a first speed when determined that the first timing has arrived;
estimating a blood pressure characteristic value representing a maximum blood pressure or a minimum blood pressure based on blood pressure characteristics information obtained during the pressurization control;
determining presence or absence of a blood pressure fluctuation based on a first blood pressure characteristic value estimated this time and a second blood pressure characteristic value up to a previous time;
carrying out a depressurization control of the cuff at a second speed slower than the first speed when determined that the blood pressure fluctuation is present; and
determining a blood pressure value of a person to be measured based on blood pressure characteristics information obtained during the depressurization control.

10. A blood pressure measurement control method for automatically executing a measurement control of a blood pressure over a plurality of times, the blood pressure measurement control method comprising the steps of:
determining whether or not a first timing corresponding to a start of a first period has arrived;
carrying out a pressurization control of a cuff at a first speed when determined that the first timing has arrived;
estimating a blood pressure characteristic value representing a maximum blood pressure or a minimum blood pressure based on blood pressure characteristics information obtained during the pressurization control;
determining presence or absence of a blood pressure fluctuation based on a first blood pressure characteristic value estimated this time and a second blood pressure characteristic value up to a previous time;
carrying out a depressurization control of the cuff at a second speed slower than the first speed when determined that the blood pressure fluctuation is present; and
determining a blood pressure value of a person to be measured based on blood pressure characteristics information obtained during the depressurization control.
